(19) United States
(12) Patent Application Publication
(10) Pub. No.: US 2014/0294791 A1
(43) Pub. Date: Oct. 2, 2014

(54) Lactobacillus paracasei subsp. paracasei, as an agent for inhibiting listeria monocytogenes in vivo infection

(71) Applicants: Tamara Smokvina, Orsay (FR); Gianfranco Grompone, Paris (FR); Sophie Legrain-Raspaud, Limours (FR); Cristel Archambaud, Jouy en Josas (FR); Marc Lecuit, Paris (FR); Pascale Cossart, Paris (FR)

(72) Inventors: Tamara Smokvina, Orsay (FR); Gianfranco Grompone, Paris (FR); Sophie Legrain-Raspaud, Limours (FR); Cristel Archambaud, Jouy en Josas (FR); Marc Lecuit, Paris (FR); Pascale Cossart, Paris (FR)

(73) Assignees: INSTITUT PASTEUR, Paris (FR); COMPAGNIE GERVAIS DANONE, Paris (FR)

(21) Appl. No.: 14/361,736

(22) PCT Filed: Dec. 1, 2011
(86) PCT No.: PCT/IB2011/055413

§ 371 Date: May 30, 2014

(51) Int. Cl. A61K 35/74; A23L 1/30

(52) U.S. Cl. CPC A61K 35/747; A23L 1/3014
USPC 424/93.45; 435/252.9

(57) ABSTRACT

The present invention relates to the use of the probiotic strain of Lactobacillus paracasei subsp. paracasei deposited at the CNCM under the reference I-3689, for inhibiting in vivo infection by Listeria monocytogenes.

The present invention relates to a novel application of the probiotic strain of Lactobacillus paracasei subsp. paracasei deposited at the CNCM under the reference I-3689. More specifically, the present invention pertains to the use of this strain for inhibiting in vivo infection by Listeria monocytogenes.

Listeria monocytogenes, a facultative anaerobe, intracellular bacterium, is the causative agent of listeriosis. L. monocytogenes is a Gram-positive bacterium, in the division Firmicutes.

L. monocytogenes is one of the most virulent foodborne pathogens, with 20 to 30 percent of clinical infections resulting in death. Invasive infection by L. monocytogenes causes the disease listeriosis. When the infection is not invasive, any illness as a consequence of infection is termed febrile gastroenteritis. The manifestations of listeriosis include septicemia, meningitis (or meningoencephalitis), encephalitis and intrauterine or cervical infections in pregnant women, which may result in spontaneous abortion (second to third trimester) or stillbirth. Surviving neonates of fetomaternal listeriosis may suffer granulomatosis infantiseptica—pyogenic granulomas distributed over the whole body, and may suffer from mental retardation.

A very large number of scientific studies have reported the beneficial effects, on the health, of certain microorganisms present in fermented foodstuffs, in particular dairy products. These microorganisms are commonly referred to as “probiotics”. According to the definition generally accepted at the current time, probiotics are: “live microorganisms which, when they are consumed in appropriate amounts, have a beneficial effect on the health of the host” (FAO/WHO report on evaluation of health and nutritional properties of probiotics in food, including powder milk containing live lactic acid bacteria; Cordoba, Argentina; Oct. 1-4, 2001).

It has been shown that the consumption of food products containing probiotic bacteria can produce favorable effects on the health, in particular through re-equilibrating the intestinal flora (especially after a dysbiosis), improving resistance to infections, and modulating the immune response.

The probiotic microorganisms used in human food are generally lactic acid bacteria belonging mainly to the Lactobacillus and Bifidobacterium genera, for example to the species Lactobacillus paracasei subsp. paracasei.

However, the beneficial effects on the health are not generally common to all the bacteria of the same genus, nor even of the same species. They are, most commonly, encountered only in certain strains; in addition, the effects observed can vary qualitatively and/or quantitatively from one probiotic strain to the other, including within the same species.

Few studies have been published concerning the role of probiotics in Listeria infection. These have been conducted mainly in vitro, or in vivo after intravenous or peritoneal infection. In particular, Coconnier et al. have described that L. acidophilus decreases Listeria adhesion and invasion in Caco-2 cells (Coconnier et al., 1993), while Corr et al. have shown that Lactobacillus and Bifidobacterium inhibit Listeria infection of C2Bbe1 cells (Corr et al., 2007). In vivo, L. casei activates innate immunity via NF-kappaB and p38 MAP kinase signaling pathways after i.v. infection with Listeria in BALB/c or SCID mice (Kim et al., 2006). More recently, dos Santos et al. have studied the effect of a Lactobacillus strain on Listeria intraperitoneal infection of germ-free mice. They describe that L. delbrueckii UFV-H2b20 bacteria favor effector responses involving TNF-α and IFN-γ, thereby protecting mice from death caused by L. monocytogenes infection (dos Santos et al., 2010).

The inventors have now studied the impact of consumption of another Lactobacillus strain, which is a particular probiotic strain of Lactobacillus paracasei subsp. Paracasei, on L. monocytogenes early steps of infection. This Lactobacillus strain was deposited, according to the Treaty of Budapest, with the CNCM (Collection Nationale de Cultures de Microorganismes [National collection of microorganism cultures], 25 rue du Docteur Roux, Paris), on Nov. 9, 2006, under number I-3689. L. paracasei CNCM I-3689 was already studied in vitro in presence of pathogenic microorganisms in culture. However, nothing is known about the effects of this strain on in vivo infection by Listeria. There is hence a need to assess the effects of L. paracasei CNCM I-3689 in a dynamic model (host) and to analyze the relationship between probiotic and host in a situation of infection.

The inventors have now demonstrated that CNCM I-3689 bacteria can protect a host from L. monocytogenes infection, through a mechanism which is different from that observed with L. delbrueckii UFV-H2b20, since the production of IFN-γ in germ-fee mice upon infection by L. monocytogenes is inferior in mice precolonized with CNCM I-3689 to that observed in non-colonized mice (see Example 2 below), whereas it is increased in mice precolonized with L. delbrueckii UFV-H2b20 (dos Santos et al., supra). The inventors have also demonstrated that this protection involves an interaction between the probiotic strain and the host, and is not the mere result of the growth inhibition which was previously observed in vitro (see the experimental part below).

A first aspect of the present invention is hence a Lactobacillus paracasei subsp. paracasei strain deposited with the CNCM under number I-3689, for use as an agent inhibiting and/or preventing in vivo infection by Listeria monocytogenes.

In particular, the strain CNCM I-3689 can be used as an agent for favoring an effector response of an individual infected by Listeria monocytogenes. Such an effector response can inhibit the spreading of L. monocytogenes in the body of the infected host, and/or inhibit the multiplication of L. monocytogenes in certain organs, and/or favor clearance of said pathogenic bacteria from the body.

It is important to note that CNCM I-3689 does not induce per se a strong inflammatory state of the host, but rather prepares the host to react more efficiently in response to an infection by L. monocytogenes. This strain is hence particularly useful as a prophylactic agent against Listeria monocytogenes infection.

As already mentioned, and illustrated in the experimental part which follows, the CNCM I-3689 strain modulates expression of interferon stimulated genes normally induced upon Listeria infection. Indeed, the signature of Listeria infection comprises an upregulation of interferon stimulated genes, which is inhibited in the presence of CNCM I-3689. Interestingly, the activation of interferon regulatory factors, which is also part of the signature of Listeria infection, is also decreased when infection occurs after oral inoculation with CNCM I-3689 bacteria. The present invention hence also pertains to the strain CNCM I-3689, for use as an agent for inhibiting the induction of interferon signaling pathway upon infection by Listeria monocytogenes, and to the same strain, for use as an agent for inhibiting the activation of interferon regulatory factors upon infection by Listeria monocytogenes.

Production of interferon gamma, especially in the spleen, is also a natural response to Listeria infection. As shown in FIG. 4, 24 h after oral infection by L. monocytogenes, this production is less elevated in mice which have previously been precolonized by Lactobacilli such as CNCM I-3689. Hence, another aspect of the present invention is the strain CNCM I-3689, for use as an agent for inhibiting the production of INF-γ in the spleen upon infection by Listeria monocytogenes.

The inventors also showed that precolonization with the CNCM I-3689 strain decreases significantly the number of Listeria within the small intestine and in the spleen of infected germ-free E16P mice. Remarkably, this was not the case when precolonization was performed with another probiotic L. casei strain, which further demonstrates the specificity of the CNCM I-3689 strain. According to another aspect, the present invention hence pertains to the CNCM I-3689 strain, for use as an agent for inhibiting the development of Listeria monocytogenes in the small intestine and/or in the spleen.

According to a preferred embodiment, the strain is administered by the oral route.

According to a particular embodiment of the present invention, the CNCM I-3689 strain is taken up regularly, for example daily, to obtain good prophylaxis properties. In a preferred embodiment, the strain is administered one to three days before the oral infection by Listeria monocytogenes.

According to another preferred embodiment, at least 2.10⁹ cells of CNCM I-3689 strain are administered in each dose, for example each day.

According to yet another preferred embodiment of the present invention, the CNCM I-3689 strain is comprised in a food preparation.

Other characteristics of the invention will also become apparent in the course of the description which follows of the biological assays which have been performed in the framework of the invention and which provide it with the required experimental support, without limiting its scope.

### LEGENDS TO THE FIGURES

FIG. 1: Experimental procedure. For each precolonization step, 2.10⁹ Lactobacillus/mouse were administered through the oral route. Infection was also performed through the oral route, with 5.10⁹ Listeria EGDe/mouse.

FIG. 2: Effect of the precolonization on Listeria infection of germ-free E16P mice. 574 : no probiotic before infection; 570 : CNCM I-3689 before infection; 568 : control Lactobacillus casei subsp. casei before infection. Independent experiments ≧3. Mouse per condition ≧3.

FIG. 3: QPCR validation of the expression of 3 host genes induced by Listeria monocytogenes (Lmo) and less induced after precolonization.

FIG. 4: IFN-γ production induced by Listeria monocytogenes (Lmo) in the small intestine (SI), in the mesenteric lymph node (MLN) and in the spleen. IFN-γ production was measured by ELISA.

### EXAMPLES

All the experimental data which follow have been obtained using the following materials and methods:

Strains and Growth Conditions

Listeria monocytogenes EGDe strain was grown in BHI medium (DIFCO) at 37° C. Lactobacillus paracasei CNCM I-3689 was grown in MRS medium (OXOID) at 37° C.

Animals

All experiments involving mice were conducted according to the Institut Pasteur guidelines for laboratory animals' husbandry. Germ-free knock-in E16P mice (Disson et al., 2008) were housed in plastic gnotobiotic isolators. Only 9-12 weeks old female mice were used for experiments. Conventional knock-in E16P mice were housed in standard conditions.

Precolonization

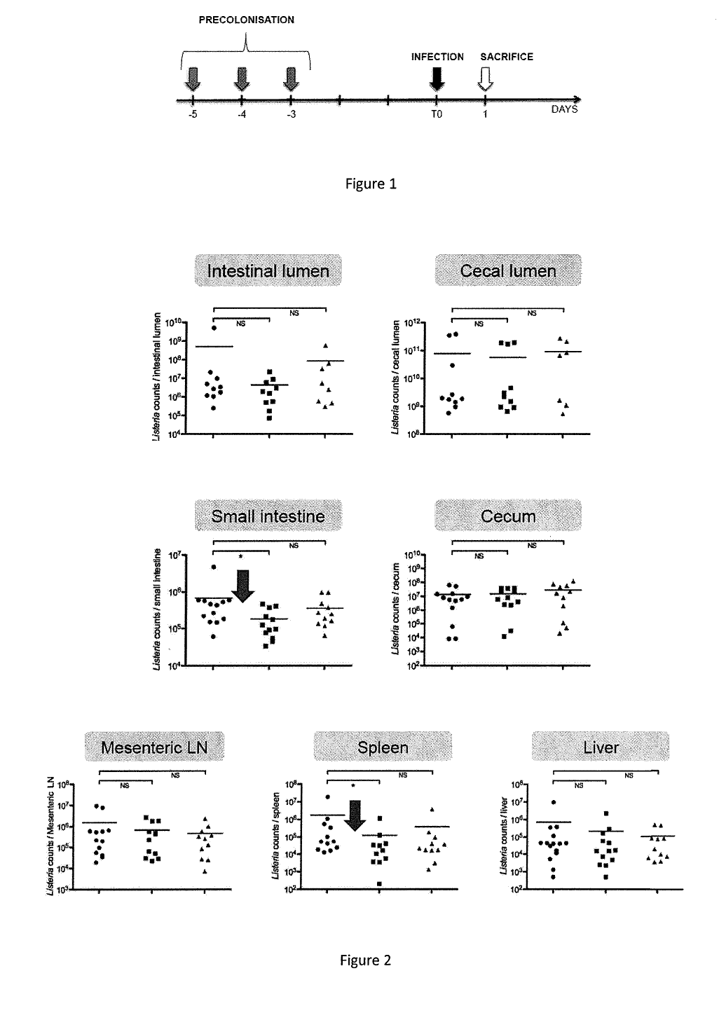

L. paracasei CNCM I-3689 overnight culture was collected and centrifuged at 4000 rpm for 15 minutes. After 3 washes in PBS, L. paracasei CNCM I-3689 pellet was resuspended in PBS at a final concentration of 1×10¹⁰ bacteria/ml. Mice were precolonized orally with 2×10⁹ bacteria diluted in 200 μl of PBS. Serial dilutions of the inoculum were plated to control the number of L. paracasei CNCM I-3689 that were inoculated in mice. This precolonization step has been repeated for 2 additional days. Mice were infected 2 days after (FIG. 1). Another probiotic strain, namely a Lactobacillus casei subsp. casei, was used as a control.

Infection

L. monocytogenes EGDe overnight culture was diluted in BHI and bacteria were grown until OD=1. Bacterial cultures were recovered and centrifuged at 4000 rpm for 15 minutes. After 3 washes in PBS, L. monocytogenes EGDe pellet was resuspended in PBS at a final concentration of 2.5×10¹⁰ bacteria/ml. Mice were infected orally with 5×10⁹ bacteria diluted in 200 μl of PBS supplemented with 300 μl of CaCO₃ (50 mg/ml). Serial dilutions of the inoculum were plated to control the number of L. monocytogenes EGDe inoculated in mice.

Bacterial Counts in Organ

Animals were sacrificed at 24 h after infection. The whole organs, i.e., small intestine, cecum, mesenteric lymph nodes, liver and spleen were separately removed. Mesenteric lymph nodes, liver and spleen were directly disrupted in 3 ml of PBS. The small intestine was removed and cut into 16 equal-sized segments (numbered 1-16; proximal-to-distal). Intestinal fragments (3-7-11-15) and cecum were washed 5 times in DMEM and incubated in DMEM containing 100 μg/ml gentamicin for 2 hours. After 5 washes in DMEM, intestinal segments and cecum were disrupted in 3 ml of PBS. Serial dilutions of all organ homogenates were plated on BHI plates and incubated for 2 days at 37° C. before CFU counts. Intestinal and cecal luminal contents were also harvested, weight and resuspended in 500 μl PBS. Serial dilutions of luminal contents were plated both on Listeria selective Oxford plates (OXOID) for Listeria counts and on MRS plates for Lactobacilli counts.

Gene Chip Analysis

RNA was extracted and purified using classical Trizol/Chloroform protocol. All samples were treated with Turbo DNAse (Ambion) according manufacturer's instructions. RNA quality was determined using Experion Automated Electrophoresis Station (Bio-Rad). Only samples reaching the quality criteria required for chip hybridization were used. RNAs were stored at −80° C. until needed. Labeled cDNA was synthesized from 200 ng total RNA using NuGEN Applause™ WT-Amp Plus ST Systems (NuGEN Technologies, San Carlos, Calif.). Labeled samples were hybridized to Affymetrix MoGene 1.0 ST GeneChips and scanned with an Affymetrix Genechip Scanner 3000, generating CEL files for each array. Three biological replicates were run for each condition. Gene-level expression values were derived from the CEL file probe-level hybridization intensities using the model-based Robust Multichip Average algorithm (RMA) (Bolstad et al., 2003). RMA performs normalization, background correction and data summarization. Analysis has been performed using the LPE test (Jain et al., 2003) and a p-value threshold of p<0.05 is used as the criterion for expression. The estimated false discovery rate (FDR) of this analysis was calculated using the Benjamini and Hochberg approach (Benjamini et al. Journal of the Royal Statistical Society Series B, (57): 289-300, 1995) in order to correct for multiple comparisons.

Q-PCR

For gene expression analysis, total eukaryotics RNAs (1 μg) was reverse transcribed using iScript cDNA synthesis (Biorad) according the manufacturer's instruction. The cDNAs were used as templates for PCR in the presence of SYBR Green PCR Master Mix (Applied Biosystems) according the manufacturer's instruction and detected using Real-Time PCR System ABI PRISM 7900HT (Applied Biosystems). Expression of individual mRNAs was normalized to expression of the GADPH gene. For miRNA expression analysis, total eukaryotics RNAs (1 μg) was reverse transcribed using miScript Reverse Transcription kit (Qiagen) according the manufacturer's instruction. The cDNAs were used as templates for PCR using miScript SYBR Green PCR kit (Qiagen) according the manufacturer's instruction and detected using Real-Time PCR System ABI PRISM 7900HT (Applied Biosystems).

Cytokine Dosage by Elisa

Cytokine level from cell culture supernatants were analyzed by classical ELISA Method. IFNγ, and IL-22 level was determined by using mouse ELISA Ready-SET-Go! kits (eBioscience, San Diego, Calif.) according to the manufacturer's instructions. Cytokine level was measured using on a Tristar LB491 luminometer (Berthold Technologies) according to the manufacturer's instructions.

Histology

Intestinal sections of zinc salt-fixed, paraffin-embedded segments (1-5-9-13) blocks were stained with hematoxylin and eosin.

### Example 1

### Impact of Probiotics on Listeria Colonization within the Host

Precolonization and infection steps were performed as shown in FIG. 1.

Both Lactobacilli colonized the intestinal and cecal content.

Some Lactobacilli have been found in deeper organs, possibly due to an enhancement of susceptibility of the germ-free mice in absence of microbiota, since preliminary experiment shows that no Lactobacilli are found in organs of conventional E16P mice.

As shown in FIG. 2, precolonisation with the CNCM I-3689 strain significantly decreased the number of Listeria within the small intestine and in the spleen of the germ-free E16P mice, in contrast to the control probiotic strain (p-value=0.0021 for the small intestine and 0.0359 for the spleen).

### Example 2

### Precolonization of Germ-Free Mice with CNCM I-3689 Lactobacilli Modulates the Host Response to Listeria Infection

Interferon gene regulation is a signature of Listeria infection in E16P mice. As shown in Table 1 below, interferon signaling in Listera infected mice is less induced after precolonization with CNCM I-3689 Lactobacilli.

TABLE 1
Indicative levels of expression of some host genes 24 h after
Listeria infection, in absence of precolonization (column A)
or after precolonization with CNCM I-3689 (column B).

| | A | B | |
|---|---|---|---|
| IRF9 | 1.8 | 1.3 | Interferon regulatory factor 9 |
| IFIT1 | 1.9 | 1.5 | Interferon-induced protein with |
| | | | tetratricopeptide repeats 1 |
| OAS1 | 2.5 | 1.2 | 2′,5′-oligoadenylate synthetase 1, 40/46 kDa |
| IRF1 | 2.6 | 1.7 | Interferon regulatory factor 1 |
| PSMB8 | 3.2 | 2.0 | Proteasome (prosome, macropain) subunit, beta |
| | | | type, 8 (large multifunctional peptidase 7) |
| TAP1 | 4.6 | 2.3 | Transporter 1, ATP-binding cassette, sub-family B |
| | | | (MDR/TAP) |
| STAT1 | 4.7 | 2.1 | Signal transducer and activator of transcription 1, |
| | | | 91 kDa |
| STAT2 | 4.9 | 2.2 | Signal transducer and activator of transcription 2, |
| | | | 113 kDa |
| IFIT3 | 5.8 | −1.3 | Interferon-induced protein with tetratricopeptide |
| | | | repeats 3 |
| MX1 | 9.1 | 2.1 | Myxovirus (influenza virus) resistance 1, interferon- |
| | | | inducible protein p78 (mouse) |

As shown in table 2 below, activation of interferon regulatory factor (IRF) in Listeria-infected mice decreases after precolonization with CNCM I-3689 Lactobacilli.

TABLE 2
Indicative levels of expression of some host genes 24 h after
Listeria infection, in absence of precolonization (column A)
or after precolonization with CNCM I-3689 (column B).

| | A | B | |
|---|---|---|---|
| TBK1 | 1.7 | 1.4 | TANK-binding kinase 1 |
| IRF9 | 1.8 | 1.3 | Interferon regulatory factor 9 |
| IFIH1 | 1.8 | −1.2 | Interferon induced with helicase C domain 1 |
| DDX58 | 1.8 | −1.1 | DEAD (Asp-Glu-Ala-Asp) box polypeptide 58 |
| ADAR | 2.1 | 1.4 | Adenosine deaminase, RNA-specific |
| ISG15 | 3.5 | −1.1 | ISG15 ubiquitin-like modifier |
| IFIT2 | 3.6 | 1.2 | Interferon-induced protein with tetratricopeptide |
| | | | repeats 2 |
| STAT1 | 4.7 | 2.1 | Signal transducer and activator of transcription 1, |
| | | | 91 kDa |
| DHX58 | 4.9 | 1.7 | DEXH (Asp-Glu-X-His) box polypeptide 58 |
| STAT2 | 4.9 | 2.2 | Signal transducer and activator of transcription 2, |
| | | | 113 kDa |
| ZBP1 | 10.1 | 5.5 | Z-DNA binding protein 1 |

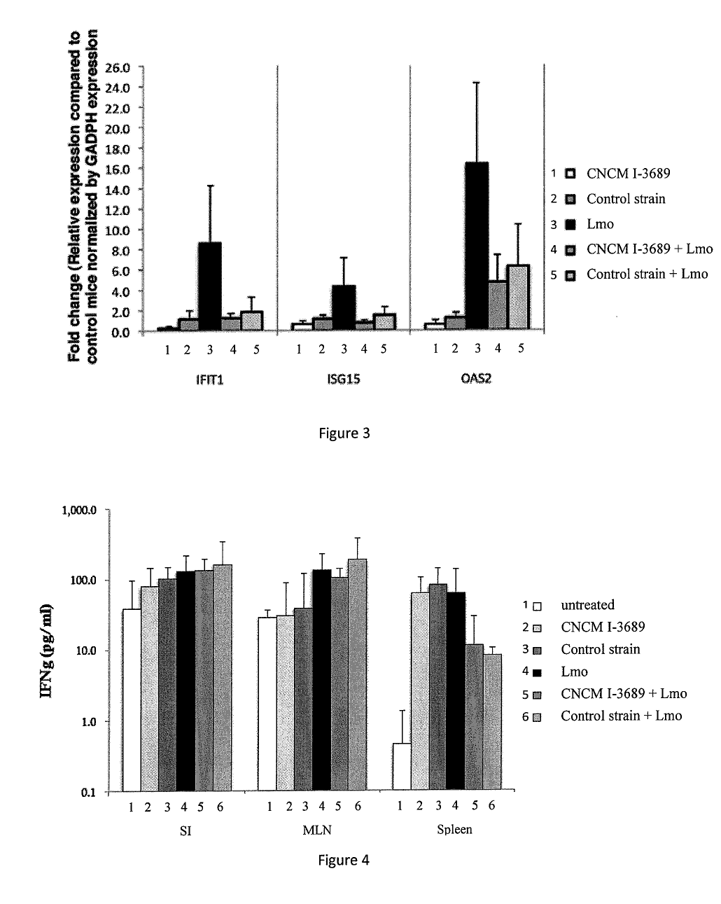

These results have been validated by quantitative PCR for three genes induced by Listeria and less induced after precolonization with CNCM I-3689 Lactobacilli (FIG. 3).

The inventors have also demonstrated, by ELISA, that IFN-γ production induced by Listeria in the spleen is lower after precolonization with Lactobacilli (FIG. 4).

### Example 3

### Impact of Precolonization with CNCM I-3689 Strain on the Induction of microRNAs in Response to L. monocytogenes Infection

Recently, Dalmasso et al. described that microbiota might modulate the host gene expression via microRNAs (Dalmasso et al.). Unpublished data from the inventors indicate that a number of microRNAs are modulated during L. monocytogenes infection. The inventors have now noted that the precolonisation step modifies the production of some of these microRNAs in response to Listeria infection.

### CONCLUSION

Altogether, these results show that precolonization with Lactobacillus paracasei CNCM I-3689 positively impacts on Listeria infection by limiting Listeria dissemination and modulating the host response both at the transcriptional and cellular level.

### REFERENCES

Bolstad, B. M., Irizarry, R. A., Astrand, M. and Speed, T. P. (2003) A comparison of normalization methods for high density oligonucleotide array data based on variance and bias. Bioinformatics, 19, 185-193.

Coconnier, M. H., Bernet, M. F., Kerneis, S., Chauviere, G., Fourniat, J. and Servin, A. L. (1993) Inhibition of adhesion of enteroinvasive pathogens to human intestinal Caco-2 cells by Lactobacillus acidophilus strain LB decreases bacterial invasion. FEMS Microbiol Lett, 110, 299-305.

Corr, S. C., Gahan, C. G. and Hill, C. (2007) Impact of selected Lactobacillus and Bifidobacterium species on Listeria monocytogenes infection and the mucosal immune response. FEMS Immunol Med Microbiol, 50, 380-388.

Dalmasso, G., Nguyen, H. T., Yan, Y., Laroui, H., Charania, M. A., Ayyadurai, S., Sitaraman, S. V. and Merlin, D. Microbiota modulate host gene expression via microRNAs. PLoS One, 6, e19293.

Disson, O., Grayo, S., Huillet, E., Nikitas, G., Langa-Vives, F., Dussurget, O., Ragon, M., Le Monnier, A., Babinet, C., Cossart, P. and Lecuit, M. (2008) Conjugated action of two species-specific invasion proteins for fetoplacental listeriosis. Nature, 455, 1114-1118.

dos Santos, L. M., Santos, M. M., de Souza Silva, H. P., Arantes, R. M., Nicoli, J. R. and Vieira, L. Q. (2010) Monoassociation with probiotic Lactobacillus delbrueckii UFV-H2b20 stimulates the immune system and protects germfree mice against Listeria monocytogenes infection. Med Microbiol Immunol, 200, 29-38.

Jain, N., Thatte, J., Braciale, T., Ley, K., O'Connell, M. and Lee, J. K. (2003) Local-pooled-error test for identifying differentially expressed genes with a small number of replicated microarrays. Bioinformatics, 19, 1945-1951.

Kim, Y. G., Ohta, T., Takahashi, T., Kushiro, A., Nomoto, K., Yokokura, T., Okada, N. and Danbara, H. (2006) Probiotic Lactobacillus casei activates innate immunity via NF-kappaB and p38 MAP kinase signaling pathways. Microbes Infect, 8, 994-1005.

1. A Lactobacillus paracasei subsp. paracasei strain deposited with the CNCM (Collection Nationale De Cultures De Microorganismes) under Accession number I-3689.

2-13. (canceled)

14. A method of inhibiting or preventing a Listeria monocytogenes infection in an individual comprising administering the Lactobacillus paracasei subsp. paracasei strain of claim 1 to the individual.

15. A method of favoring an effector response in an individual infected by Listeria monocytogenes comprising administering the Lactobacillus paracasei subsp. paracasei strain of claim 1 to the individual.

16. A method of inhibiting induction of an interferon signaling pathway in an individual caused by infection by Listeria monocytogenes comprising administering the Lactobacillus paracasei subsp. paracasei strain of claim 1 to the individual.

17. A method of inhibiting the activation of interferon regulatory factors in an individual caused by infection by Listeria monocytogenes comprising administering the Lactobacillus paracasei subsp. paracasei strain of claim 1 to the individual.

18. A method of decreasing INF-γ production in the spleen of an individual caused by infection by Listeria monocytogenes comprising administering the Lactobacillus paracasei subsp. paracasei strain of claim 1 to the individual.

19. A method of inhibiting proliferation of Listeria monocytogenes in the small intestine of an individual comprising administering the Lactobacillus paracasei subsp. paracasei strain of claim 1 to the individual.

20. A method of inhibiting proliferation of Listeria monocytogenes in the spleen of an individual comprising administering the Lactobacillus paracasei subsp. paracasei strain of claim 1 to the individual.

21. A method of prophylaxis against Listeria monocytogenes infection in an individual comprising administering a prophylactically effective amount of the Lactobacillus paracasei subsp. paracasei strain of claim 1 to the individual.

22. The method of claim 14, wherein the Lactobacillus paracasei subsp. paracasei strain is administered daily.

23. The method of claim 14, wherein the Lactobacillus paracasei subsp. paracasei strain is administered by the oral route.

24. The method of claim 14, wherein the Lactobacillus paracasei subsp. paracasei strain is administered one to three days before oral infection by Listeria monocytogenes.

25. The method of claim 14, wherein at least 2×10⁹ cells of the Lactobacillus paracasei subsp. paracasei strain are administered each day.

26. The method of claim 14, wherein the Lactobacillus paracasei subsp. paracasei strain is present in a food product.
